(19) United States
(12) Patent Application Publication
(10) Pub. No.: US 2017/0086800 A1
(43) Pub. Date: Mar. 30, 2017

(54) METHODS AND DEVICES FOR TISSUE BIOPSY

(71) Applicant: BOSTON SCIENTIFIC SCIMED, INC., Maple Grove, MN (US)

(72) Inventors: Martyn G. Folan, Loughrea (IE); Thomas M. Keating, Galway (IE); Thomas Holly, Galway (IE); Martin Hynes, Galway (IE); Hugh Mulcahy, Dublin (IE)

(21) Appl. No.: 15/274,571

(22) Filed: Sep. 23, 2016

Related U.S. Application Data
(60) Provisional application No. 62/232,754, filed on Sep. 25, 2015.

(51) Int. Cl. A61B 10/02

(52) U.S. Cl. CPC A61B 10/0266; A61B 10/0283

(57) ABSTRACT

Medical devices and methods are disclosed in the present application. In one illustrative example a tissue removal device may comprise an elongate shaft defining a lumen and having a proximal end and a distal end, wherein a proximal portion of the elongate shaft has a first diameter and a distal portion of the elongate shaft has a second diameter, and wherein the first diameter is less than the second diameter and an elongate member disposed at least partially within the elongate shaft, the elongate member having a proximal end and a distal end, the distal end of the elongate member comprising a cap. In some further embodiments, the elongate member may be axially translatable between a proximal position and a distal position, and the cap may form a closed receptacle with the distal portion of the elongate shaft when the elongate member is in the proximal position.

### CROSS-REFERENCE TO RELATED APPLICATIONS

This application claims priority under 35 U.S.C. §119 to U.S. Provisional Application Ser. No. 62/232,754, filed Sep. 25, 2015, the entirety of which is incorporated herein by reference.

### TECHNICAL FIELD

This disclosure relates to medical devices or tissue biopsy, and more particularly, to medical devices for tissue biopsy and maintaining integrity of the biopsy sample.

### BACKGROUND

In many patients, vessels can become constricted for a variety of reasons, resulting in a stricture or narrowing of the vessel. For example, plaque may build-up in a location within a vessel, a growth may form on a wall of a vessel, or the wall of the vessel may become inflamed. In some cases it may be desirable to biopsy tissue at or near the stricture site in order to identify the cause of the stricture. A medical device may be inserted into the vessel and positioned proximate to the stricture. A part of the medical device may then scrape, cut, or otherwise remove tissue at the biopsy site to be retrieved for analysis. However, it may be difficult to obtain enough tissue for useful analysis or to remove the biopsied tissue from the patient without contaminating the sample.

### SUMMARY

This disclosure relates to medical devices or tissue biopsy, and more particularly, to medical devices for tissue biopsy and maintaining integrity of the biopsy sample. In a first illustrative example, a tissue removal device may comprise an elongate shaft defining a lumen and having a proximal end and a distal end, wherein a proximal portion of the elongate shaft may have a first diameter and a distal portion of the elongate shaft may have a second diameter, and wherein the first diameter is less than the second diameter and an elongate member disposed at least partially within the elongate shaft, the elongate member having a proximal end and a distal end, the distal end of the elongate member comprising a cap. In at least some additional embodiments, the elongate member may be axially translatable between a proximal position and a distal position, and the cap may form a closed receptacle with the distal portion of the elongate shaft when the elongate member is in the proximal position.

Additionally, or alternatively, in any of the above embodiments with respect to the first illustrative embodiment, the distal portion of the elongate shaft may be removable from the proximal portion of the elongate shaft.

Additionally, or alternatively, in any of the above embodiments with respect to the first illustrative embodiment, the cap may be removable from the elongate member.

Additionally, or alternatively, in any of the above embodiments with respect to the first illustrative embodiment, the distal portion of the elongate shaft may screw onto the elongate shaft.

Additionally, or alternatively, in any of the above embodiments with respect to the first illustrative embodiment, the distal portion of the elongate shaft may comprise a frangible connection.

Additionally, or alternatively, in any of the above embodiments with respect to the first illustrative embodiment, the cap may be rotatable relative to the elongate shaft.

Additionally, or alternatively, in any of the above embodiments with respect to the first illustrative embodiment, the cap may extend distally beyond the distal end of the elongate shaft.

Additionally, or alternatively, in any of the above embodiments with respect to the first illustrative embodiment, a proximally facing edge of the cap may comprise a cutting edge.

Additionally, or alternatively, in any of the above embodiments with respect to the first illustrative embodiment, a distal edge of the distal portion of the elongate shaft may have a cutting edge.

Additionally, or alternatively, in any of the above embodiments with respect to the first illustrative embodiment, the cap may be a hollow cap.

Additionally, or alternatively, in any of the above embodiments with respect to the first illustrative embodiment, the elongate member may be a hollow elongate member defining a guidewire lumen, and wherein the cap may further comprise a distal guidewire port.

Additionally, or alternatively, in any of the above embodiments with respect to the first illustrative embodiment, the device may further comprise a suction mechanism for providing suction through the lumen of the elongate shaft.

Additionally, or alternatively, in any of the above embodiments with respect to the first illustrative embodiment, the suction mechanism may comprise a vacuum reservoir connected to the proximal portion of the elongate shaft and a plunger slidably receiving within the vacuum reservoir moveable between a proximal position and a distal position to provide suction through the lumen of the elongate shaft.

Additionally, or alternatively, in any of the above embodiments with respect to the first illustrative embodiment, the elongate member may extend through the plunger of the suction mechanism.

Additionally, or alternatively, in any of the above embodiments with respect to the first illustrative embodiment, the device may further comprise a balloon member disposed proximate the distal portion of the elongate shaft.

Additionally, or alternatively, in any of the above embodiments with respect to the first illustrative embodiment, the elongate shaft may further define an inflation lumen connected to an interior of the balloon member.

Additionally, or alternatively, in any of the above embodiments with respect to the first illustrative embodiment, the balloon may extend only partway around the elongate shaft.

Additionally, or alternatively, in any of the above embodiments with respect to the first illustrative embodiment, when the elongate shaft is disposed within a vessel and the balloon is inflated, the balloon may be configured to displace the elongate shaft towards one side of the vessel.

Additionally, or alternatively, in any of the above embodiments with respect to the first illustrative embodiment, the cap may be disposed on an opposite side of the elongate shaft as the balloon member.

Additionally, or alternatively, in any of the above embodiments with respect to the first illustrative embodiment, the cap may be a semi-circular cap.

Additionally, or alternatively, in any of the above embodiments with respect to the first illustrative embodiment, the cap may be rotatable relative to the elongate shaft.

Additionally, or alternatively, in any of the above embodiments with respect to the first illustrative embodiment, the distal portion of the elongate shaft having the second diameter has a semi-circular shape.

In a second illustrative embodiment, a tissue removal device may comprise an elongate shaft defining a lumen and having a proximal end and a distal end and an elongate member disposed at least partially within the elongate shaft, the elongate member having a proximal end and a distal end, the distal end of the elongate member comprising a cap. In at least some additional embodiments the elongate member may be axially translatable between a proximal position and a distal position, and the cap may form a closed receptacle with a distal portion of the elongate shaft when the elongate member is in the proximal position. Additionally, a distal portion of the elongate shaft may be removable from the elongate shaft.

Additionally, or alternatively, in any of the above embodiments with respect to the second illustrative embodiment, the distal portion of the elongate shaft may screw onto the elongate shaft.

Additionally, or alternatively, in any of the above embodiments with respect to the second illustrative embodiment, the distal portion of the elongate shaft may comprise a frangible connection.

Additionally, or alternatively, in any of the above embodiments with respect to the second illustrative embodiment, the cap may be rotatable relative to the elongate shaft.

Additionally, or alternatively, in any of the above embodiments with respect to the second illustrative embodiment, the device may further comprise a balloon member disposed proximate the distal end of the elongate shaft.

Additionally, or alternatively, in any of the above embodiments with respect to the second illustrative embodiment, the cap may be a hollow cap.

Additionally, or alternatively, in any of the above embodiments with respect to the second illustrative embodiment, the elongate shaft may further define an inflation lumen in communication with an interior of the balloon member.

Additionally, or alternatively, in any of the above embodiments with respect to the second illustrative embodiment, the balloon may extend only partway around the elongate shaft.

Additionally, or alternatively, in any of the above embodiments with respect to the second illustrative embodiment, when the elongate shaft is disposed within a vessel and the balloon is inflated, the balloon may be configured to displace the elongate shaft towards one side of the vessel.

Additionally, or alternatively, in any of the above embodiments with respect to the second illustrative embodiment, a proximally facing edge of the cap may comprise a cutting edge.

Additionally, or alternatively, in any of the above embodiments with respect to the second illustrative embodiment, a distal edge of the distal portion of the elongate shaft may have a cutting edge.

Additionally, or alternatively, in any of the above embodiments with respect to the second illustrative embodiment, the elongate member may be a hollow elongate member defining a guidewire lumen, and wherein the cap may further comprise a distal guidewire port.

Additionally, or alternatively, in any of the above embodiments with respect to the second illustrative embodiment, the device may further comprise a suction mechanism for providing suction through the lumen of the elongate shaft.

Additionally, or alternatively, in any of the above embodiments with respect to the second illustrative embodiment, the suction mechanism may comprise a vacuum reservoir connected to the proximal portion of the elongate shaft and a plunger slidably receiving within the vacuum reservoir moveable between a proximal position and a distal position to provide suction through the lumen of the elongate shaft.

Additionally, or alternatively, in any of the above embodiments with respect to the second illustrative embodiment, the elongate member may extend through the plunger of the suction mechanism.

In a third illustrative embodiment, a method of tissue biopsy may comprise inserting a biopsy device into a vessel of a patient. The biopsy device may comprise an elongate shaft defining a lumen and having a proximal end and a distal end, wherein a proximal portion of the elongate shaft has a first diameter and a distal portion of the elongate shaft has a second diameter, and wherein the first diameter is less than the second diameter and an elongate member disposed at least partially within the elongate shaft, the elongate member having a proximal end and a distal end, the distal end of the elongate member comprising a cap. In some embodiments, the elongate member may be axially translatable between a proximal position and a distal position and the cap may form a closed receptacle with the distal portion of the elongate shaft when the elongate member is in the proximal position. The method may further comprise positioning the distal end of the elongate shaft proximate a biopsy site and translating the elongate member from the distal position to the proximal position to cut tissue at the biopsy site and retain the tissue within the closed receptacle.

Additionally, or alternatively, in any of the above embodiments with respect to the third illustrative embodiment, the distal portion of the elongate shaft may be removable from the elongate shaft and the cap may be removable from the elongate member. In these embodiments, the method may further comprise removing the distal portion of the elongate shaft and the cap forming the receptacle from the biopsy device.

Additionally, or alternatively, in any of the above embodiments with respect to the third illustrative embodiment, the biopsy site may be located in a bile duct of the patient.

The above summary of the present disclosure is not intended to describe each embodiment or every implementation of the present disclosure. Advantages and attainments, together with a more complete understanding of the disclosure, will become apparent and appreciated by referring to the following detailed description and claims taken in conjunction with the accompanying drawings.

### BRIEF DESCRIPTION OF THE DRAWINGS

The disclosure may be more completely understood in consideration of the following detailed description of various embodiments in connection with the accompanying drawings, in which:

FIG. 1 is a perspective view of a biopsy device in accordance with various embodiments of the present disclosure;

FIG. 2A is a close-up of a distal end of the biopsy device of FIG. 1 where a cap the biopsy device is in a distal position;

FIG. 2B is a close-up of a distal end of the biopsy device of FIG. 1 where a cap the biopsy device is in a proximal position;

FIG. 3 is a perspective view of a distal end of a biopsy device including a screw connection;

FIG. 4 is a perspective view of a distal end of a biopsy device including a frangible connection;

FIG. 5 side view of another exemplary biopsy device, in accordance with aspects of the present disclosure;

FIG. 6 is a cross-section of an end of an elongate shaft of the biopsy device of FIG. 1 as viewed in a plane parallel to line A-A;

FIG. 7 is a cross-section of a cap of the biopsy device of FIG. 1 as viewed in a plane parallel to line B-B;

FIG. 8 is a perspective view of an alternative cap of a biopsy device, in accordance with aspects of the present disclosure;

FIG. 9 is a view of an end of a biopsy device according to aspects of the present disclosure, including a vacuum suction mechanism;

FIG. 10 is another view of the vacuum suction mechanism of FIG. 9, where the vacuum suction mechanism has been activated;

FIG. 11 is a perspective view of an end of a biopsy device according to aspects of the present disclosure including a vacuum suction mechanism where the vacuum suction mechanism has been activated;

FIGS. 12A-12C depict an example operation of the biopsy device of FIG. 1;

FIGS. 13A and 13B are side and top views of another exemplary biopsy device including a balloon, in accordance with aspects of the present disclosure;

FIG. 14A is a cross-section of the biopsy device of FIGS. 13A and 13B, as viewed from a plane parallel to line C-C;

FIG. 14B is a cross-section of the biopsy device of FIGS. 13A and 13B, as viewed from a plane parallel to line D-D;

FIGS. 15A and 15B depict an example operation of the biopsy device of FIGS. 13A and 13B.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges and/or values pertaining to various components, features and/or specifications are disclosed, one of skill in the art, incited by the present disclosure, would understand desired dimensions, ranges and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended to be only exemplary. Selected features of any illustrative embodiments may be incorporated into any other described embodiments unless clearly stated to the contrary.

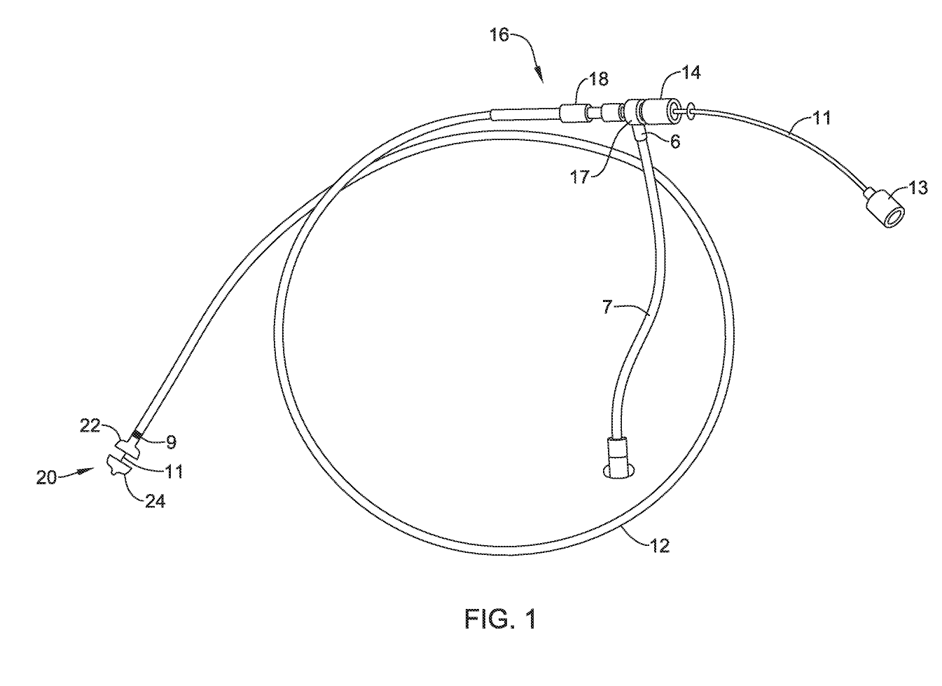

FIG. 1 shows an exemplary biopsy device 10 in accordance with various embodiments of the present disclosure. In some cases, biopsy device 10 may comprise elongate shaft 12 disposed about elongate member 11. Elongate shaft 12 may be, for example, a catheter shaft and may have a length and an outside diameter to enable biliary tract insertion and navigation. For example, elongate shaft 12 may have a length of about 20-250 cm and an outside diameter of approximately 1-10 French, depending upon the desired application. In other cases, biopsy device 10 may be a microcatheter device that is adapted and/or configured for use within smaller anatomies of the patient, and elongate shaft 12 may have an appropriately smaller outside diameter. For instance, in some applications, biopsy device 10 may be used to navigate to targets sites located in tortuous and narrow vessels such as to sites within the neurovascular system, certain sites within the coronary vascular system, to sites within the peripheral vascular system such as superficial femoral, popliteal, or renal arteries, or any number of locations within the biliary tract. In some cases, the target site is a neurovascular site and may be located within a patient's brain, which is accessible only via a tortuous vascular path. However, it is contemplated that biopsy device 10 may be used in other target sites within the anatomy of a patient. An exemplary catheter having a shaft that may be similar to elongate shaft 12 and utilized in accordance with the various embodiments as described herein is shown and described in U.S. Pat. No. 8,182,465, which is incorporated herein by reference in its entirety for all purposes.

Elongate shaft 12 may generally extend from proximal portion 16 and proximal end 18 toward distal portion 20. Although elongate shaft 12 may have a circular cross-sectional shape, it should be understood that elongate shaft 12 can have other cross-sectional shapes or combinations of shapes without departing from the scope of the disclosure. For example, the cross-sectional shape of the generally tubular elongate shaft 12 may be oval, rectangular, square, triangular, polygonal, and the like, or any other suitable shape, depending upon the desired characteristics.

In some cases, biopsy device 10 may include manifold 14 connected to proximal end 18 of elongate shaft 12. Manifold 14 may include hub 17 and/or other structures to facilitate connection to other medical devices (e.g., syringe, stopcocks, Y-adapter, etc.) and to provide access to one or more lumens defined within elongate shaft 12. In some cases, hub 17 may include port 6 which provides individual access to one or more lumens extending through at least a portion of biopsy device 10. Manifold 14 is shown with elongate tube 7 connected to port 6. In some embodiments, elongate tube 7 may be connected to vacuum source to provide aspiration during use of biopsy device 10. Some example lumens that may extend through elongate shaft 12 include at least one guidewire lumen and one or more inflation lumens. However, in at least some embodiments, tubular elongate shaft 12 may only include a single lumen. Any lumens that do extend through catheter 10 may terminate at or near distal portion 20 of elongate shaft 12, as will be described with respect to other figures. In other cases, hub 17 may have two ports, three ports, or any other number of ports, and in some embodiments may have one port for each lumen extending through elongate shaft 12.

Biopsy device 10, and more particularly elongate shaft 12, may be made from a polymer (some examples of which are disclosed below), a metal-polymer composite, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

In at least some embodiments, portions or all of biopsy device 10 and/or elongate shaft 12, may also be loaded with, made of, or otherwise include a radiopaque material. Radiopaque materials are understood to be materials capable of producing a relatively bright image on a fluoroscopy screen or another imaging technique during a medical procedure. For example, marker band 9 may be embedded or attached to elongate shaft 12 proximate distal end 20, as shown in FIG. 1. This relatively bright image aids the user of cat biopsy device 10 in determining locations of portions of the devices. Some examples of radiopaque materials can include, but are not limited to, gold, platinum, palladium, tantalum, tungsten alloy, polymer material loaded with a radiopaque filler (e.g., barium sulfate, bismuth subcarbonate, etc.), and the like.

In some embodiments, a coating may be applied to the exterior surface of the biopsy device 10 and/or elongate shaft 12. For example, a lubricious, a hydrophilic, a protective, or other type of coating may be applied over portions or all of the biopsy device 10 and/or elongate shaft. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

The coating and/or biopsy device 10 and/or elongate shaft 12, may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present invention.

Elongate member 11 may include distal end 13 and may generally extend through elongate shaft 12, terminating in cap 24. In some embodiments, cap 24 may be attached to elongate member 11 and extend beyond the distal end of elongate member 11, while in other embodiments may be a part of elongate member 24. Elongate member 11 may be a shaft similar to elongate shaft 12 and made from similar materials. For instance, elongate member 11 may be a relatively flexible hollow tube. In these embodiments, elongate member 11 may generally have a single lumen configured to receive a guidewire. However, in other embodiments, elongate member 11 may include additional lumens.

In alternative embodiments, elongate member 11 may be a guidewire. An example guidewire that may be similar to elongate member 11 is shown and described in U.S. Pat. No. 7,955,272, which is incorporated herein by reference in its entirety for all purposes. In such embodiments, elongate member 11 may be made formed of a super-elastic material such as the alloys of nickel and titanium, commonly known as Nitinol. While Nitinol is the most common super-elastic material, any of a variety of other super-elastic materials may be used for elongate member 11. Other alloys by chemical name include; CuAlNi, CuSn, CuZn, InTi, NiAl, FePt, MnCu, and FeMnSi. A detailed discussion of super-elastic alloys and their processing is presented in U.S. Pat. No. 4,925,445 which is herein incorporated by reference.

In addition to super-elastic materials, linear-elastic materials may be used. Linear-elastic materials are described in U.S. Pat. No. 5,238,004, which is also incorporated herein by reference. In general, linear-elastic materials are composed of the same alloys above. However, different material processing strategies are used to provide a wire which has many of the important characteristics of a super-elastic material without some of the difficulties related to machining, specifically grinding. As such, elongate member 11 may preferably be formed of a linear-elastic alloy of nickel-titanium.

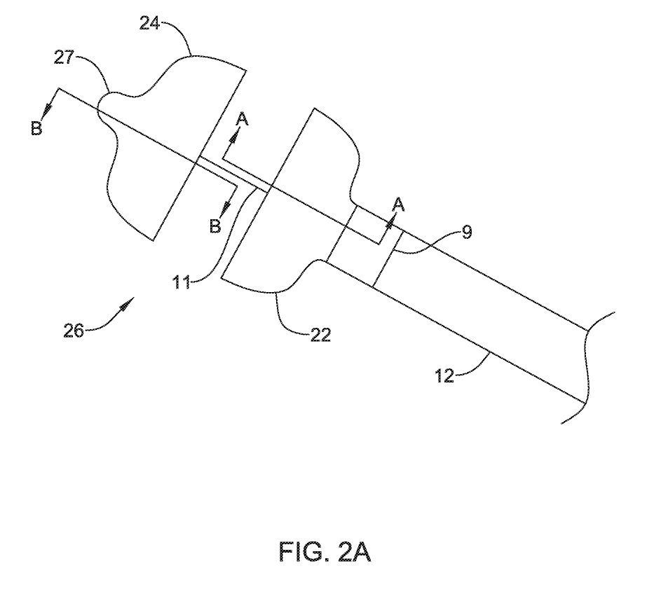

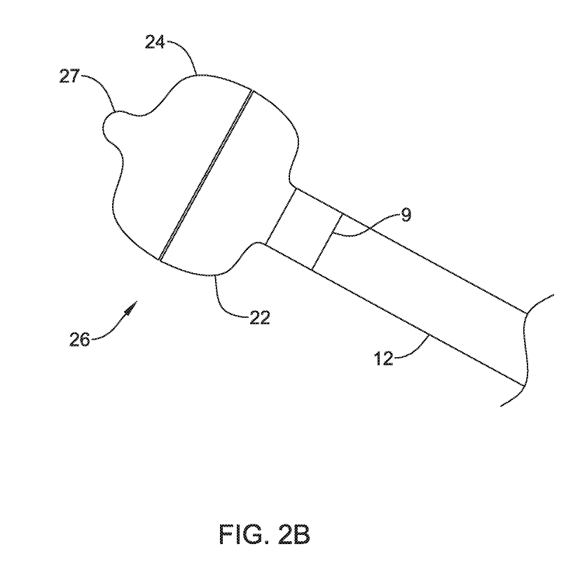

FIGS. 2A and 2B depict a close-up of distal portion 20 of biopsy device 10, including expanded end 22 and cap 24. As can be seen in FIGS. 2A and 2B, in some embodiments of elongate shaft 12, elongate shaft 12 may have a first diameter along a proximal portion and second diameter along a distal portion. For instance, distal portion 20 may include expanded end 22 which has a greater outer diameter than the rest of elongate shaft 12. Cap 24 may similarly have an outer diameter greater than the rest of elongate member 11, and in some cases the proximal portion of elongate shaft 12. In at least some embodiments, the outer diameters of expanded end 22 and cap 24 may be similar or may match.

However, it is not necessary in all embodiments of biopsy device 10 for elongate shaft 12 to have an expanded end. In other embodiments, distal portion 20 may not include any expanded end such that elongate shaft 12 has the same diameter along the entire shaft. In these embodiments, the outer diameter of cap 24 may match this diameter of elongate shaft 12.

Elongate member 11 may be axially translatable with respect to elongate shaft 12. For instance, elongate member 11 may be moveable between a proximal position, shown in FIG. 2A, and a distal position, shown in FIG. 2B. Cap 24 and expanded end 22, together, may form receptacle 26. For instance, cap 24 and expanded end 22 may be generally hollow such that, when elongate member 11 is in the proximal position, expanded end 22 and cap 24 are brought together to form a receptacle for holding biopsied tissue, as will be described in more detail below.

Additionally, although expanded end 22 and cap 24 are shown as generally circular components, in other embodiments, expanded end 22 and/or cap 24 may be shaped differently. For instance, expanded end 22 and/or cap 24 may be rectangular, oval-shaped, triangular, or other shapes, and, when brought together, form a similarly shaped receptacle. In some embodiments, expanded end 22 and/or cap 24 may not extend all the way around elongate member 11 and elongate shaft 12. For instance, expanded end 22 and/or cap 24 may be half-circular shaped. In such embodiments, expanded end 22 and/or cap 24 may extend outward from elongate member 11 and/or elongate shaft 12 only about 180 degrees around elongate member 11 and/or expanded end 22. Of course, in still other embodiments, expanded end 22 and/or cap 24 make take other partial shapes and only extend outward from elongate member 11 and/or expanded end 22 between about 45 degrees to about 270 degrees around elongate member 11 and/or elongate shaft 12.

In embodiments where elongate member 11 is a hollow tube configured to receive a guidewire, cap 24 may have guidewire port 27 located at a distal end of cap. This may allow the entirety of biopsy device 10 to be slid over a guidewire to position biopsy device 10 at a desired biopsy location. Once in position, elongate member 11 may be translated axially with respect to elongate shaft 12 without also translating the guidewire to close receptacle 26. In other embodiments, elongate member 11 may not include guidewire port 27 and elongate member 11 may be a guidewire. In these embodiments where elongate member 11 is the guidewire, biopsy device 10 may include a proximal temporary connection (not shown) to couple elongate member 11 and elongate shaft 12 such that advancement of elongate member 11 into the patient also advances elongate shaft 12. Where elongate member 11 is not the guidewire but also does not include guidewire port 27, the guidewire may be inserted into elongate member 11 until contacting cap 24. Elongate member 11 may then be advanced through the patient by advancement of the guidewire. Once in position, elongate shaft 12 may be advanced over elongate member 11.

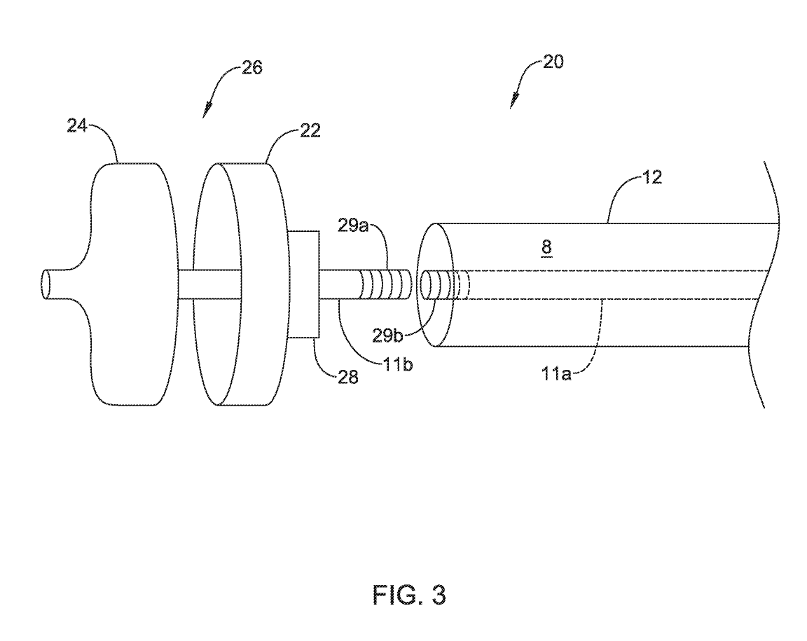

In some further embodiments of biopsy device 10, receptacle 26 may be removable from the rest of biopsy device 10. For instance, FIG. 3 depicts biopsy device 10 including threaded connection 29a, 29b, which allows receptacle 26 to be removed from the rest of biopsy device 10. In this embodiment, elongate member 11 may comprise at least two portions, elongate member portion 11a and elongate member portion 11b. A distal end of elongate member portion 11a may include threads 29b, while elongate member portion 11b may include threads 29a. Threads 29a and 29b may cooperate with each other to connect elongate member portion 11a to elongate member portion 11b.

As shown in FIG. 3, expanded end 22 may be a separate component from elongate shaft 12. In some of these embodiments, expanded end 22 may include connector 28. Connector 28 may be sized to be received within lumen 8 of elongate shaft 12. The distal end of elongate shaft 12 may be heat treated or shrunk to create a connection between expanded end 22 and elongate shaft 12. However, the connection may not be so secure that expanded end 22 may not still be separated from elongate shaft 12. In other embodiments, connector 28 may include threads that may cooperate with threads on elongate shaft 12 (not shown) in order to connect expanded end 22 to elongate shaft 12, as described with respect to elongate member portion 11a and elongate member portion 11b.

Such a removable connection may allow for easier processing of any biopsied tissue. For instance, once tissue is received within receptacle 26, and biopsy device 10 is withdrawn from the patient, receptacle 26 may be removed from biopsy device 10 and may be delivered directly to a lab for analysis. A new receptacle may then be attached to biopsy device 10, device 10 inserted back into the patient, and a new biopsy sample may be taken.

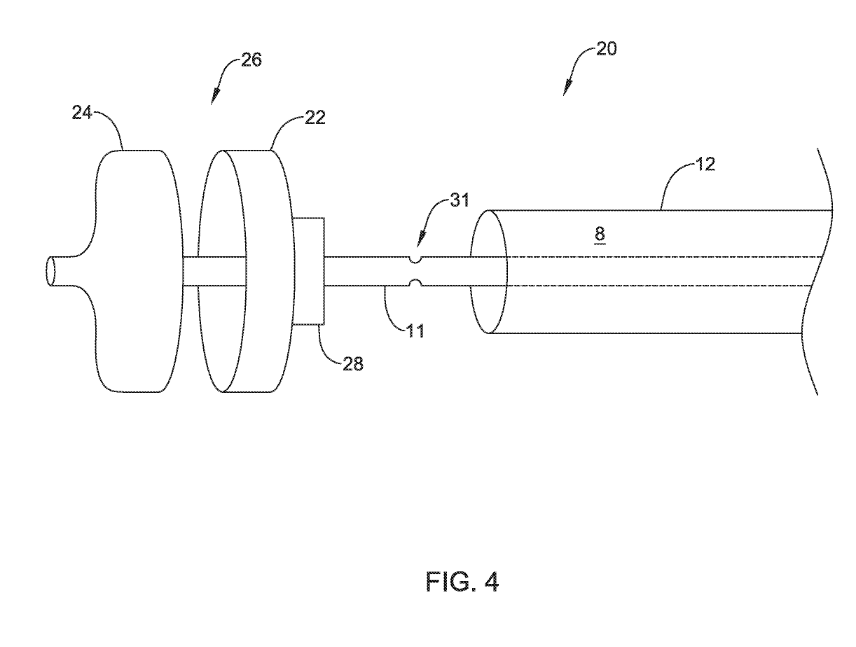

FIG. 4 shows an alternative embodiment of biopsy device 10 including a removable receptacle. In the embodiment of FIG. 4, receptacle 26 may be frangibly connected to the rest of biopsy device 10, as shown by frangible connection 31. In some embodiments, frangible connection 31 may be a weakened portion of elongate member 11 where elongate member 11 may be easily separated. Frangible connection 31 may be formed, for instance, by removing material from an outer wall of elongate member 11. The material may be removed by any means, such as cutting or laser trimming. In other embodiments, frangible connection 31 may be formed by crimping elongate member 11 or otherwise mechanically weakening elongate member 11. In still other embodiments, when elongate member 11 is formed, frangible connection 11 may be formed by adjusting an extrusion process to create a thin-walled region. Although in the embodiment of FIG. 4 another receptacle may not be attached to biopsy device 10 once the first receptacle is removed, the device still benefits from many other advantages, such as providing a secure enclosure to transport the biopsied material.

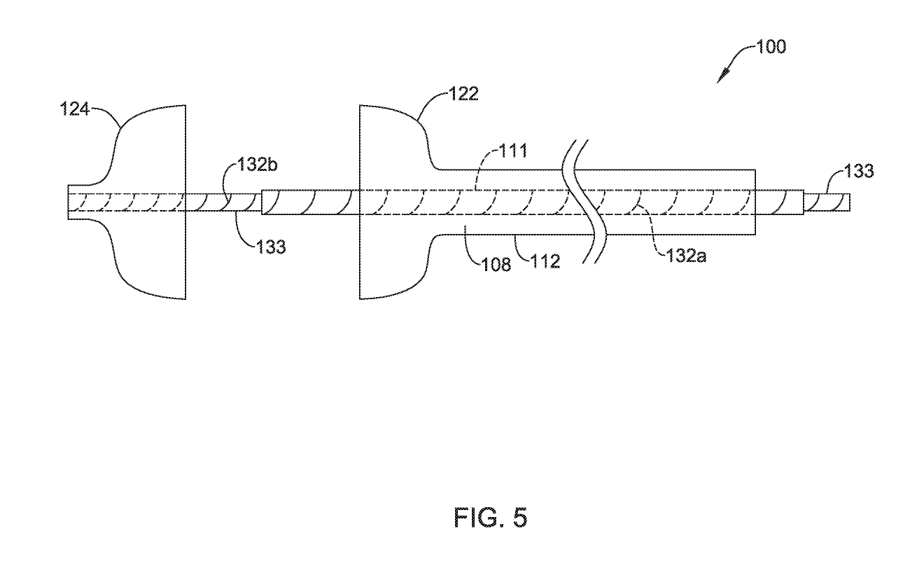

FIG. 5 depicts another example biopsy device 100. Biopsy device 100 may be similar to biopsy device 10 in many respects. For instance, biopsy device 100 may include an elongate shaft 112, an elongate member 111, and expanded end 122 and cap 124 forming receptacle 126. However, biopsy device 100 may further include shaft 133.

Additionally, elongate member 111 may be hollow and include threads 132a extending along the entire length of elongate member 111. Threads 132a may cooperate with threads 132b of shaft 133 to allow advancement or retraction of cap 124 by rotation of shaft 133. So, cap 124 may be translated from a distal position to a proximal position to engage expanded end 122 to form a receptacle by rotating shaft 133 in a first direction and may be translated distally away from expanded end 122 by rotating shaft 133 in the opposite direction.

In the embodiment of FIG. 5, expanded end 122 may be permanently attached to elongate shaft 112, or may be formed as part of elongate shaft 112. Accordingly, receptacle 126 may not be detachable from the rest of biopsy device 100 in at least some of these embodiments. In other embodiments, however, elongate shaft 112, elongate member 111, and shaft 133 may all have frangible connections that align when cap 124 is brought together with expanded end 122 to form receptacle 126. When in this position, each of elongate shaft 112, elongate member 111, and shaft 133 may be disconnected at their frangible connections to detach receptacle 126.

The embodiment of FIG. 5 may have the additional advantage of maintaining the position of cap 124 with respect to expanded end 122. For instance, after the distal end of biopsy device 100 containing cap 124 and expanded end 122 has been removed from the patient, the receptacle formed by cap 124 and expanded end 122 may more easily maintain a seal, as cap 124 and expanded end 122 may not be separated simply by pulling the two components apart. Additionally, the rotational movement of cap 124 when cap 124 is being translated may help to sever tissue when an edge of cap 124 is sharpened to cut tissue, as will be described in more detail below.

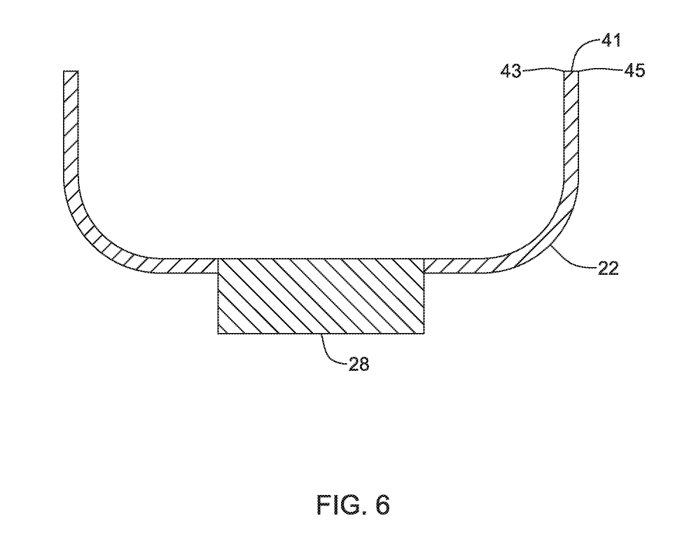

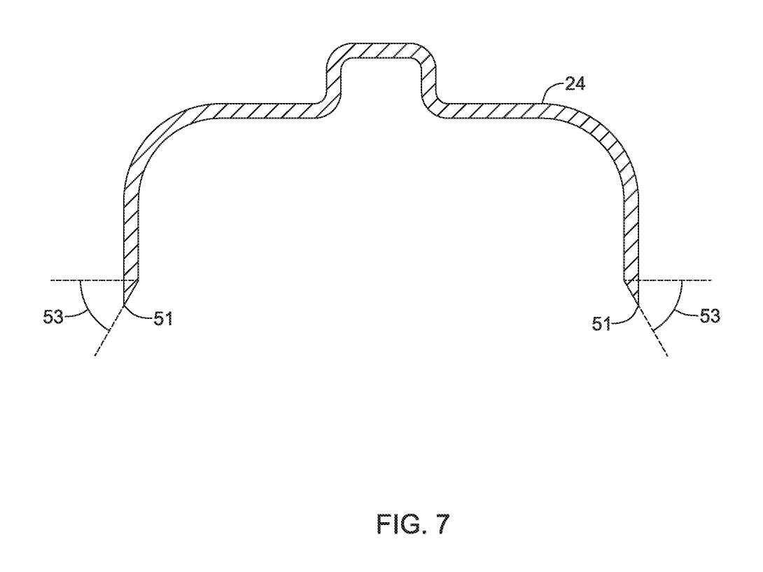

FIGS. 6 and 7 are cross-sections of expanded end 22 taken along line A-A of FIG. 2A and of cap 24 taken along line B-B of FIG. 2B, respectively. As shown, in some embodiments, expanded end 22 may have relatively flat distal-facing edge 41 while cap 24 may have a sharpened proximal facing edge 51. As cap 24 is translated with respect to expanded end 22, sharpened edge 51 may cut through tissue of the patient to create a tissue biopsy.

In some embodiments, sharpened edge 51 may be aligned directly with flat edge 41 such that, when cap 24 is in the proximal position and forming a closed receptacle with expanded end 22, sharpened edge 51 directly contacts flat edge 41 in a hammer-and-anvil type configuration. However, in other embodiments, sharpened edge 51 may be aligned with flat edge 41 such that, as cap 24 is translated towards expanded end 22, sharpened edge 51 may slide past outer edge 45 such that expanded end 22 is partially received within cap 24. In still other embodiments, sharpened edge 51 may be aligned with flat edge 41 such that, as cap 24 is translated towards expanded end 22, sharpened edge 51 may slide past inner edge 43 such that cap 24 is partially received within expanded end 22.

As seen in FIG. 7, sharpened edge 51 may form an angle 53 with to the rest of cap 24. In some embodiments, angle 53 may range from about 16 degrees to about 30 degrees, and in more specific examples may be about 17 degrees, about 18 degrees, about 19 degrees, about 20 degrees, or any other suitable value.

Of course, in other embodiments, cap 24 may have a flat proximally facing edge and expanded end 22 may have a distally facing cutting edge. In these embodiments, the description of cap 24 and expanded end 22 with respect to FIGS. 6 and 7 may be reversed.

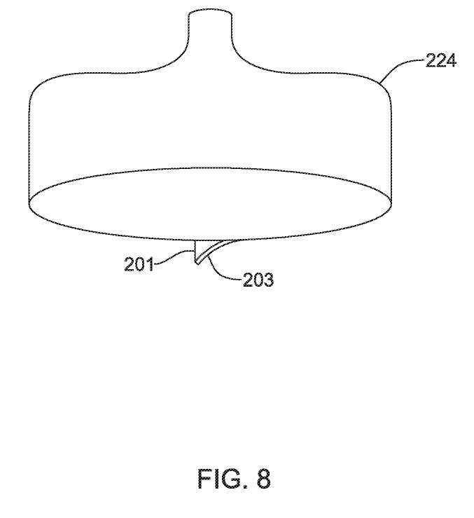

FIG. 8 shows another example embodiment of a cap, cap 224, that may be used with any of the biopsy devices disclosed herein. Cap 224 may be similar to cap 224, except that cap 224 may further include cutting flange 201. Cutting flange 201 may extend in a proximal facing direction from the proximal edge of cap 224. In these embodiments, instead of a proximally facing edge of cap 224 being sharpened to cut tissue, cutting flange 201 may include bevel 203 which produces a sharp, cutting edge on cutting flange 201. Cap 224 may be best employed where cap 224 is axially translatable in a rotational manner, such as that discussed with respect to biopsy device 100 of FIG. 5.

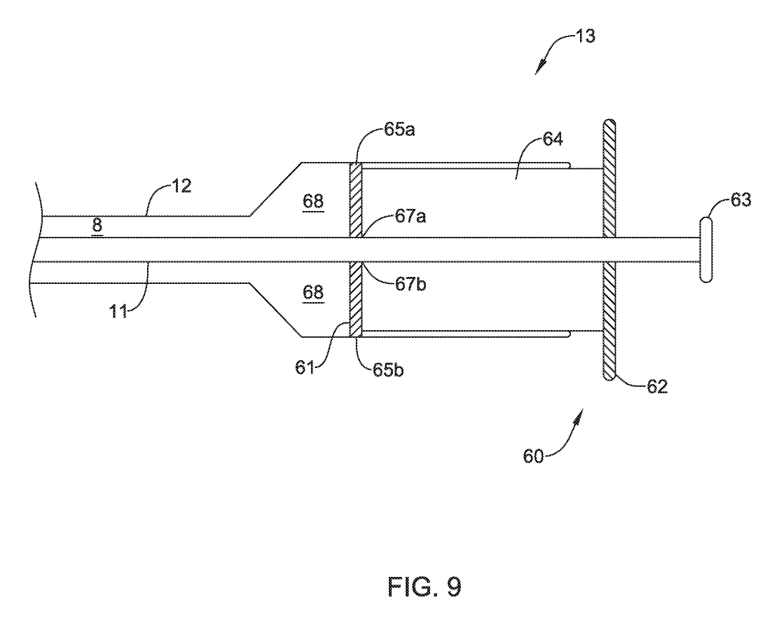

In some embodiments, distal end 13 of elongate member 11 be similar to the rest of elongate member 11. Alternatively, distal end 13 may include a grasping component by which a user may more easily manipulate elongate member 11. In at least some embodiments, distal end 13 may further include suction mechanism 60, as shown in FIG. 9. Suction mechanism 60 may allow for suction to be applied through lumen 8 of elongate shaft 12 and through expanded end 22, drawing tissue towards expanded end 22 and cap 24 to aid in the tissue biopsy.

In some embodiments, suction mechanism 60 may be similar to a standard syringe. For instance, suction mechanism 60 may comprise a plunger 64, coupled to a sealing ring 61. Sealing ring 61 may seal against an outer wall of suction mechanism 60, as shown at points 65a and 65b. Additionally, in some embodiments, elongate member 11 may extend through suction mechanism 60 as shown in FIG. 9. In such examples, sealing ring 61 may further seal against elongate member 11, as shown at points 67a and 67b. Sealing ring 61 may allow elongate member 11 to be translated with respect to elongate shaft 12, while still maintaining a seal against elongate member 11.

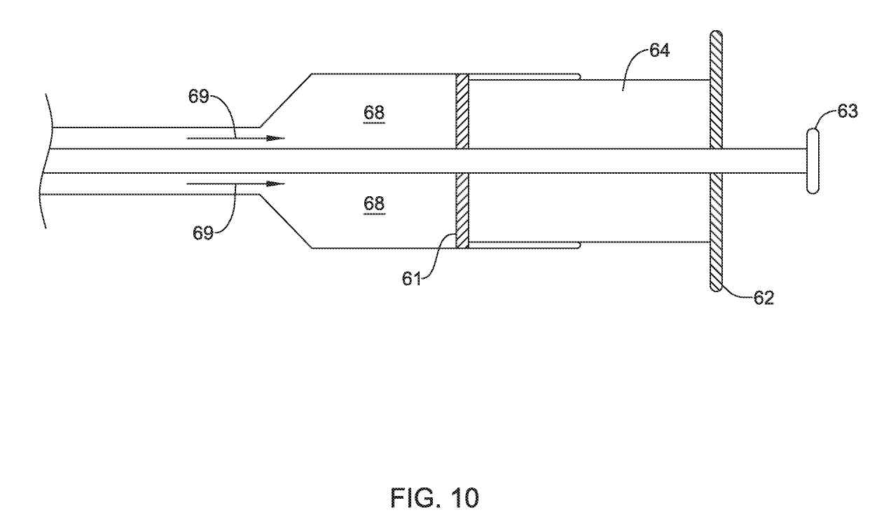

Suction mechanism 60 may additionally include handle 62. Handle 62 may allow a user to easily grasp suction mechanism 60 and translate plunger 64 within vacuum reservoir 68. Since sealing ring 61 is sealed against the outer wall of suction mechanism 60, as a user translates plunger 64 in a distal direction, enlarging vacuum reservoir 68, as shown in FIG. 10, suction may occur in the direction of arrows 69.

FIGS. 9 & 10 also depict handle 63. Handle 63 may be attached to a distal portion of elongate member 11 in order to allow a user to more easily manipulate elongate member 11. For instance, a user may manipulate elongate member 11 to axially translate elongate member 11 with respect to elongate shaft 12 in order to open or close receptacle 26.

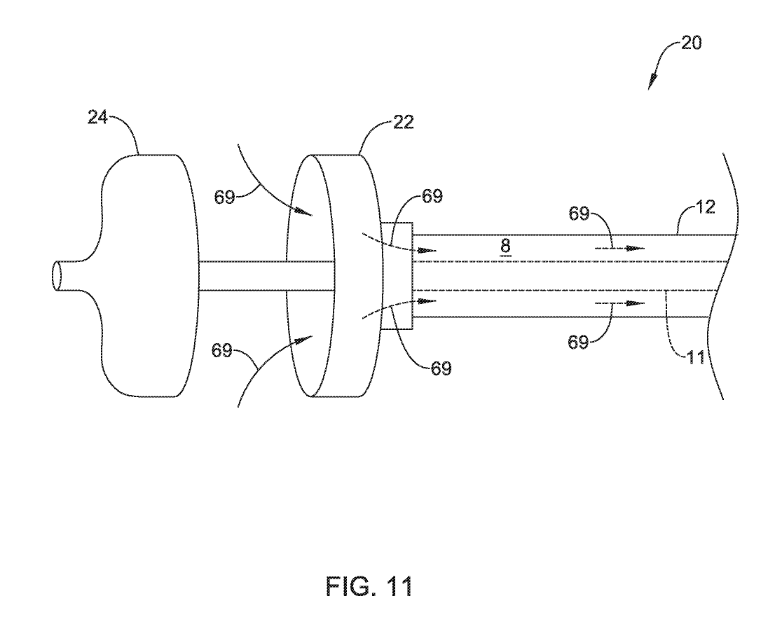

FIG. 11 depicts distal end 20 as plunger 64 is moved in the distal direction. In the embodiments described in FIGS. 9 and 10, lumen 8 runs all the way from vacuum reservoir 68 to distal end 20. Accordingly, when plunger 64 is moved in the distal direction and suction occurs, the suction may be translated down lumen 8 and through expanded end 22, as indicated by arrows 69 in FIG. 11. This suction may help draw tissue in toward expanded end 22 and cap 24 to aid in biopsy of the tissue.

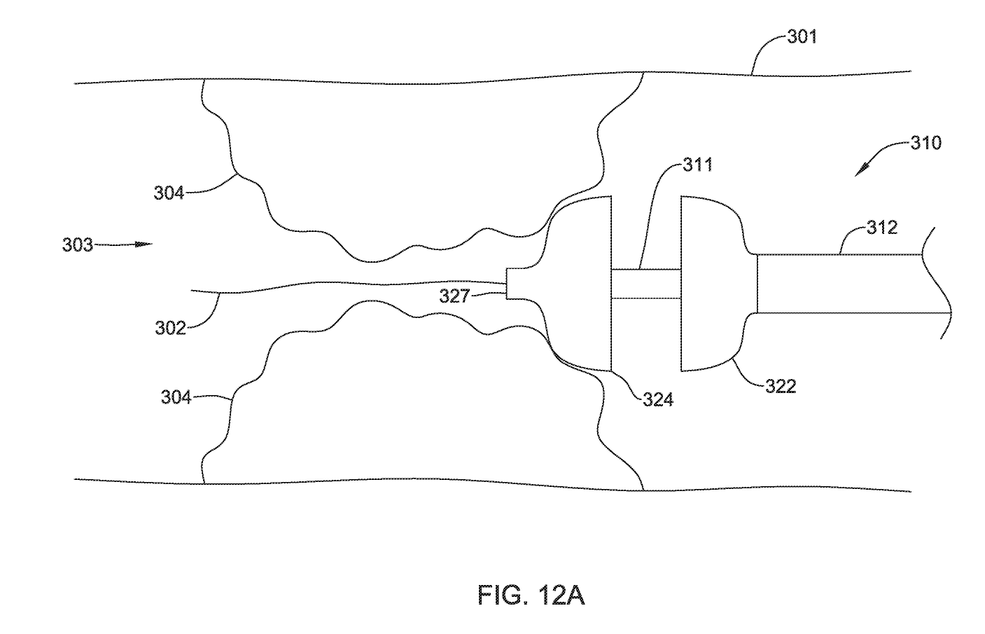

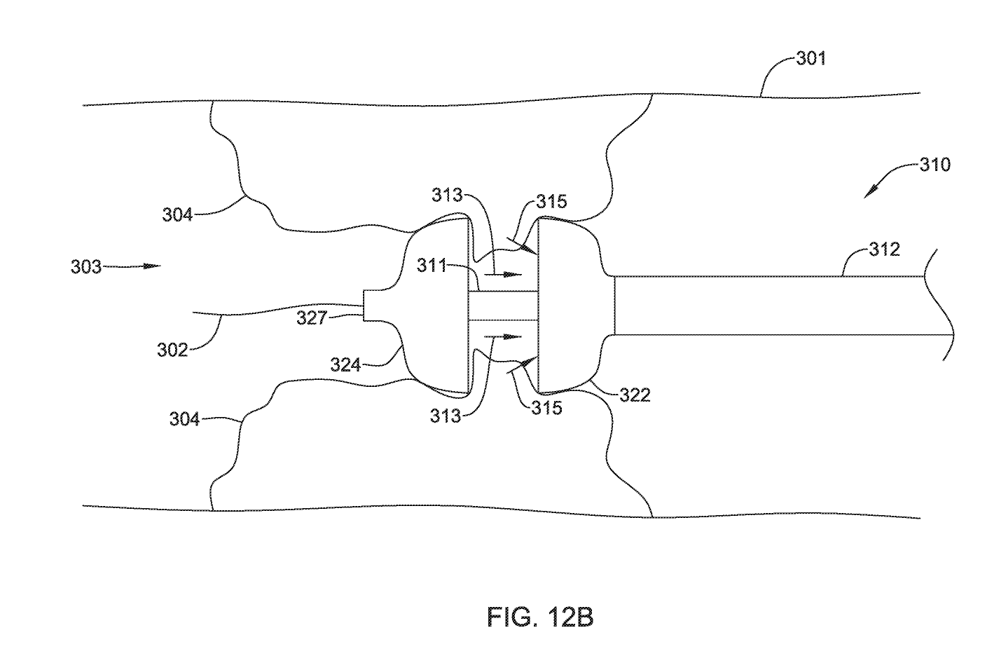

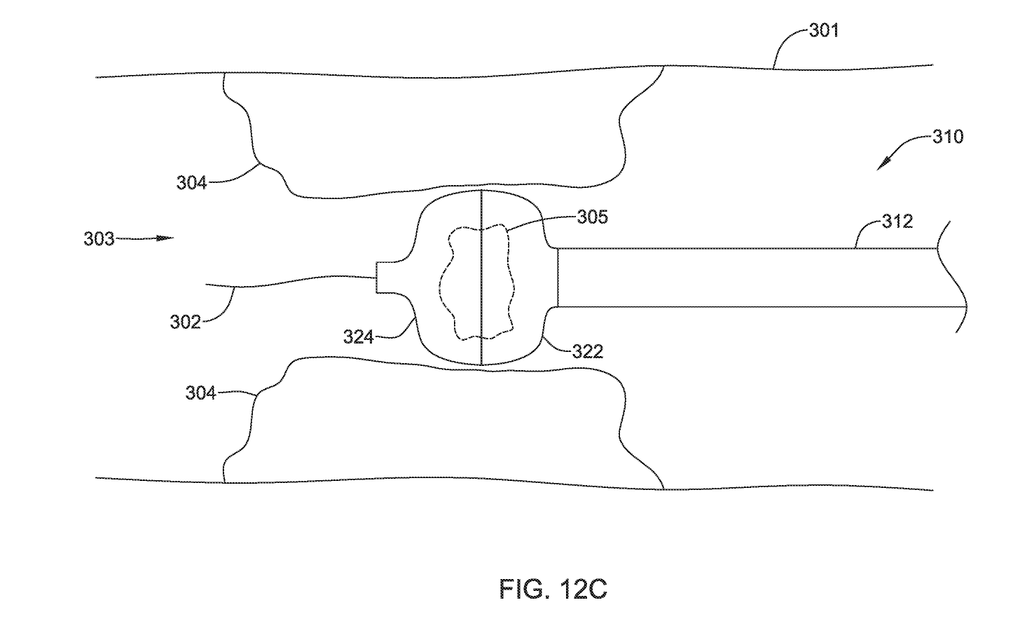

FIGS. 12A-C depicts an example biopsy procedure using biopsy device 310, which may be similar to biopsy device 10 of FIG. 1. FIG. 12A depicts stricture 303 within vessel 301. Stricture 303 may be causes by tissue 304 growing within vessel 301, plaque buildup, tissue surrounding vessel 301 pressing vessel 301 closed, or other various reasons. In at least some embodiments, tissue 303 may be cancerous or pre-cancerous and the collected biopsy may be analyzed to determine if tissue 303 is cancerous or pre-cancerous. As mentioned previously, in some embodiments, vessel 301 may be a portion of the biliary tract. However, in other embodiments, vessel 301 may represent other vessels of a patient.

A physician may begin the biopsy procedure by positioning guidewire 302 within or proximate stricture 303. The physician may then slide biopsy device 310 over guidewire 302 via distal guidewire port 327 and also position biopsy device 310 proximate stricture 303, as seen in FIG. 12A. Although, in other embodiments where biopsy device 310 does not include distal guidewire port 327, the physician may position biopsy device 310 proximate stricture 303 by inserting guidewire 302 into elongate member 311 and pushing guidewire 302 and biopsy device 310 through the patient to stricture 303. In still other embodiments, elongate member 311 may be guidewire 302, and the physician may position biopsy device 310 proximate stricture 303 by pushing elongate member 311 through the patient and then sliding elongate shaft 312 over guidewire 302.

Once biopsy device 310 is proximate stricture 303, the physician may translate cap 324 in the proximal direction, as shown by arrows 313 in FIG. 12B. In embodiments where biopsy device 310 includes a suction mechanism, suction, as shown by arrows 315, may be applied just before and/or during retraction of cap 324 toward expanded end 322. This may help to draw tissue 304 into the space between expanded end 322 and cap 324.

As cap 324 is retracted toward expanded end 322, cap 324 and/or expanded end 322 may sever tissue 304 and retain severed tissue 305, as shown in FIG. 12C, within the space defined by expanded end 322 and cap 324. The entire device may then be retracted out of the patient. In embodiments where expanded end 322 and cap 324 are removable from the rest of biopsy device 310, expanded end 322 and cap 324 may then be removed, while still containing tissue 305.

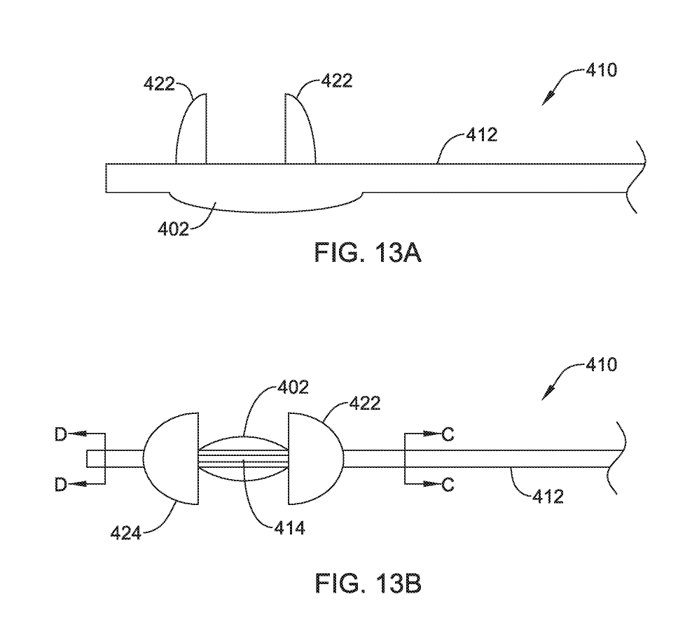

FIGS. 13A and 13B depict a distal end of yet another example biopsy device, biopsy device 410. FIG. 13A depicts a side view of the distal end of biopsy device 410. Biopsy device 410 may be similar to any of the biopsy devices disclosed herein. However, biopsy device 410 may further include a balloon 402 attached to the distal end of biopsy device 410, proximate expanded end 422 and cap 424. In the embodiments of biopsy device 410, expanded end 422 and cap 424 may only extend partially around elongate shaft 412 rather than all the way around elongate shaft 412. Similarly, balloon 402 may only extend partially around elongate shaft 412, and may generally be disposed on the opposite side of elongate shaft 412 than expanded end 422 and cap 424.

FIG. 13B depicts a top view of the distal end of biopsy device 410. As can be seen in FIG. 13B, in some embodiments, biopsy device 410 may further include slot 414 disposed proximate expanded end 422 and cap 424. Slot 414 may allow for a member, such as a member similar to elongate members 11 and 111 as described with respect to other biopsy devices, to extend through elongate shaft 412 and attach to cap 424. Slot 414 may additionally allow for translation of cap 424 with respect to expanded end 422. For example, as the member (not shown) is retracted through slot 414, cap 424 will be pulled along slot 414 toward expanded end 422.

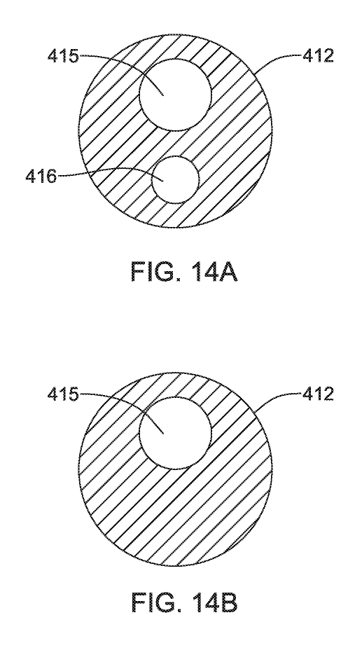

Since biopsy device 410 includes balloon 402, elongate shaft 412 of biopsy device 410 may include more than one lumen. For instance, biopsy device 410 may include at least a guidewire lumen and an inflation lumen. The configuration of lumens of biopsy device 410 may be seen more specifically in FIGS. 14A and 14B.

FIG. 14A depicts a cross-section of elongate shaft 412 taken along the line C-C in FIG. 13B. As can be seen in FIG. 14A, elongate shaft 412 may include at least guidewire lumen 415 and inflation lumen 416 running through a portion of elongate shaft 412. Inflation lumen 416 may connect to an interior of balloon 402. FIG. 14B depicts a cross-section of elongate shaft 412 taken along the line D-D in FIG. 13B. As can be seen, distal of balloon 402, elongate shaft 412 may only include guidewire lumen 415. Accordingly, inflation lumen 416 may terminate at balloon 402.

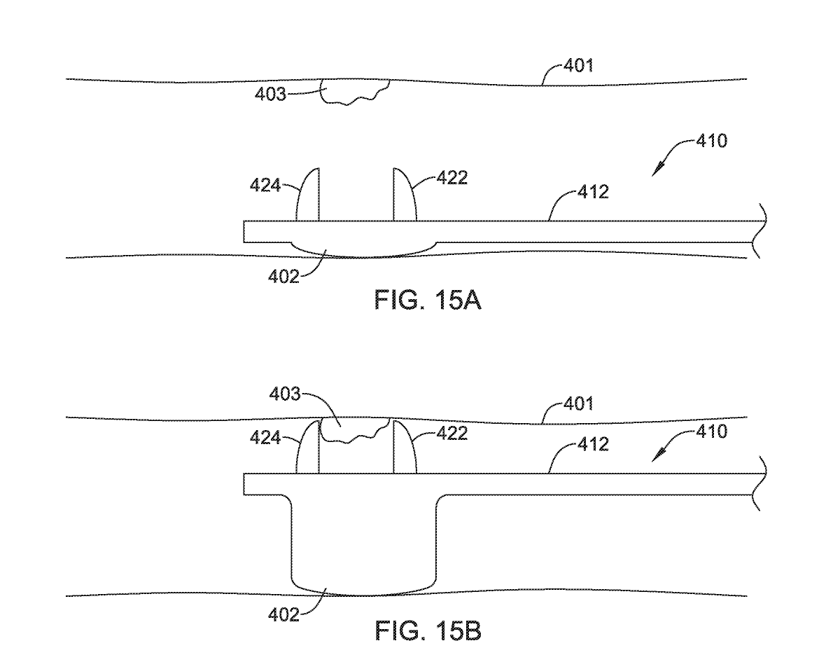

FIGS. 15A and 15B depict operation of biopsy device 410. FIG. 15A depicts biopsy device 410 situated within vessel 401. In some instances, tissue 403, which a physician may desire to biopsy, is relatively small or located only along a portion of vessel 410. In these situations, it may be difficult to maneuver the distal end of a biopsy device to accurately biopsy tissue 403.

Balloon 402 of biopsy device 410, however, may assist a physician in accurately positioning biopsy device 410. For instance, balloon 402 may be configured to displace elongate shaft 412 toward one side of vessel 401, as shown in FIG. 15B. As one example, once expanded end 422 and cap 424 are in position within vessel 401 proximate tissue 403, a physician may inflate balloon 402 using any standard inflation media. As balloon 402 expands, balloons 402 may displace elongate shaft 412, and therefore expanded end 422 and expanded end 22 and/or cap 424 toward a wall of vessel 401, positioning expanded end 422 and cap 424 adjacent to tissue 403. Once in the position of FIG. 15B, a user may translate cap 424 toward expanded end 422 to sever at least a portion of tissue 403 creating a biopsy sample, as described with respect to previous figures. The physician may then deflate balloon 402 by withdrawing the inflation media and extracting biopsy device 410, including the biopsied tissue 403.

Those skilled in the art will recognize that the present disclosure may be manifested in a variety of forms other than the specific embodiments described and contemplated herein. Specifically, the various features described with respect to the various embodiments and figures should not be construed to be applicable to only those embodiments and/or figures. Rather, each described feature may be combined with any other feature in various contemplated embodiments, either with or without any of the other features described in conjunction with those features. Accordingly, departure in form and detail may be made without departing from the scope of the present disclosure as described in the appended claims.

What is claimed:

1. A tissue removal device comprising:
an elongate shaft defining a lumen and having a proximal end and a distal end, wherein a proximal portion of the elongate shaft has a first diameter and a distal portion of the elongate shaft has a second diameter, and wherein the first diameter is less than the second diameter; and
an elongate member disposed at least partially within the elongate shaft, the elongate member having a proximal end and a distal end, the distal end of the elongate member comprising a cap;
wherein the elongate member is axially translatable between a proximal position and a distal position, and
wherein the cap forms a closed receptacle with the distal portion of the elongate shaft when the elongate member is in the proximal position.

2. The device of claim 1, wherein the distal portion of the elongate shaft is removable from the proximal portion of the elongate shaft.

3. The device of claim 1, wherein the cap is removable from the elongate member.

4. The device of claim 1, wherein the cap extends distally beyond the distal end of the elongate shaft.

5. The device of claim 1, wherein a proximally facing edge of the cap comprises a cutting edge.

6. The device of claim 1, wherein a distal edge of the distal portion of the elongate shaft is a cutting edge.

7. The device of claim 1, wherein the cap is a hollow cap.

8. The device of claim 1, wherein the elongate member is a hollow elongate member defining a guidewire lumen, and wherein the cap further comprises a distal guidewire port.

9. The device of claim 1, further comprising a suction mechanism for providing suction through the lumen of the elongate shaft.

10. A tissue removal device comprising:
an elongate shaft defining a lumen and having a proximal end and a distal end; and
an elongate member disposed at least partially within the elongate shaft, the elongate member having a proximal end and a distal end, the distal end of the elongate member comprising a cap;
wherein the elongate member is axially translatable between a proximal position and a distal position,
wherein the cap forms a closed receptacle with a distal portion of the elongate shaft when the elongate member is in the proximal position, and
wherein a distal portion of the elongate shaft is removable from the elongate shaft.

11. The device of claim 10, wherein the distal portion of the elongate shaft screws onto the elongate shaft.

12. The device of claim 10, wherein the distal portion of the elongate shaft comprises a frangible connection.

13. The device of claim 10, wherein the cap is rotatable relative to the elongate shaft.

14. The device of claim 10, further comprising a balloon member disposed proximate the distal end of the elongate shaft.

15. The device of claim 14, wherein the elongate shaft further defines an inflation lumen in communication with an interior of the balloon member.

16. The device of claim 14, wherein the balloon extends only partway around the elongate shaft.

17. The device of claim 14, wherein, when the elongate shaft is disposed within a vessel and the balloon is inflated, the balloon is configured to displace the elongate shaft towards one side of the vessel.

18. A method of tissue biopsy comprising:
inserting a biopsy device into a vessel of a patient, the biopsy device comprising:
an elongate shaft defining a lumen and having a proximal end and a distal end, wherein a proximal portion of the elongate shaft has a first diameter and a distal portion of the elongate shaft has a second diameter, and wherein the first diameter is less than the second diameter; and
an elongate member disposed at least partially within the elongate shaft, the elongate member having a proximal end and a distal end, the distal end of the elongate member comprising a cap;
wherein the elongate member is axially translatable between a proximal position and a distal position, and
wherein the cap forms a closed receptacle with the distal portion of the elongate shaft when the elongate member is in the proximal position;
positioning the distal end of the elongate shaft proximate a biopsy site; and
translating the elongate member from the distal position to the proximal position to cut tissue at the biopsy site and retain the tissue within the closed receptacle.

19. The method of claim 18, wherein:
the distal portion of the elongate shaft is removable from the elongate shaft, and the cap is removable from the elongate member; and
the method further comprises removing the distal portion of the elongate shaft and the cap forming the receptacle from the biopsy device.

20. The method of claim 18, wherein the biopsy site is located in a bile duct of the patient.
